(19) United States
(12) Patent Application Publication
(10) Pub. No.: US 2021/0290357 A1
(43) Pub. Date: Sep. 23, 2021

(54) FLOW REDUCTION STENT-GRAFT

(71) Applicant: W. L. Gore & Associates, Inc., Newark, DE (US)

(72) Inventors: Jeffrey B. Duncan, Flagstaff, AZ (US); Paul D. Goodman, Flagstaff, AZ (US)

(21) Appl. No.: 17/260,289

(22) PCT Filed: Jul. 23, 2019
(86) PCT No.: PCT/US2019/043043

§ 371 Date: Jan. 14, 2021

Related U.S. Application Data
(60) Provisional application No. 62/702,717, filed on Jul. 24, 2018.

(51) Int. Cl. A61F 2/07

(52) U.S. Cl. CPC A61F 2/07; A61F 2250/0039

(57) ABSTRACT

Various aspects of the present disclosure are apparatuses, systems and methods for altering blood flow in a vessel of a patient. The apparatuses, systems and methods may include restricting blood flow within a first side branch vessel to reduce flow into the first side branch and increase flow into one or more arteries distal to the first side branch vessel.

### CROSS-REFERENCE TO RELATED APPLICATION

This application is a national phase application of PCT Application No. PCT/US2019/043043, internationally filed on Jul. 23, 2019, which claims the benefit of Provisional Application No. 62/702,717, filed Jul. 24, 2018, both of which are incorporated herein by reference in their entireties for all purposes.

### TECHNICAL FIELD

The present disclosure relates to systems, medical devices, and methods for treating heart failure and/or other cardiovascular diseases. More specifically, the disclosure relates to removing buildup of excess fluid that typically results from poorly perfused kidneys.

### BACKGROUND

Patients experiencing heart failure may have a buildup of excess fluid in the body. The excess fluid buildup may increase fluid accumulation in the interstitial space and worsen a patient's symptoms and quality of life. Excess fluid (or hypervolemia) is the leading cause of hospitalization for heart failure patients (approximately 1,000,000 per year in the United States).

Treatment of the excess fluid buildup may be treated pharmaceutically by diuretics (or other pharmaceutical agents). However, a patient may experience drug resistance, unwanted side effects, inappropriate dosing, or other issues such as failure to comply with medicine directives. Non-pharmaceutical options, such as implantable device solutions that provide an alternative to or augment pharmaceutical efficacy by influencing renal function, may be beneficial to avoid these and other issues in treatment of buildup of excess fluid in the body. Similarly, chronic high blood pressure (hypertension) can also be managed pharmaceutically by diuretics (or other anti-hypertensive pharmaceutical agents). In addition, other disease states may result in hypotension, reduced cardiac output, and poor renal function. Insofar as the kidneys play a central role in regulating systemic blood pressure and fluid homeostasis, non-pharmaceutical options, such as implantable device solutions that provide an alternative to or augment pharmaceutical efficacy by influencing renal function, may provide an alternative means of managing the fluid imbalance resulting from chronic disease states such as heart failure, hypertension and other disease states.

### SUMMARY

In one example (“Example 1), a method of altering blood flow in a vessel of a patient includes delivering an implantable medical device including a stent element and a graft component attached to at least a portion of the stent element within the vessel; and arranging the implantable medical device to restrict blood flow within a first side branch vessel to reduce flow into the first side branch and increase flow into one or more arteries distal to the first side branch vessel and supplying an organ of the patient.

In another example (“Example 2”), further to the method of Example 1, the organ is one of kidneys, brain, pancreas, or liver.

In another example (“Example 3”), further to the method of Example 1, the method includes arranging the implantable medical device to restrict blood flow within the first side branch vessel includes reducing flow into the first side branch to increase flow into one or both renal arteries of the patient improve kidney perfusion.

In another example (“Example 4”), further to the method of Example 3, the method includes arranging the implantable medical device to restrict blood flow within the first side branch vessel includes covering the first side branch vessel arranged proximal to a renal artery ostia.

In another example (“Example 5”), further to the method of any one of Examples 1-4, the method includes the arranging the implantable device includes arranging a perfusable portion of the implantable medical device adjacent to the first side branch vessel.

In another example (“Example 6”), further to the method of Example 5, the method includes arranging the perfusable portion adjacent to the first side branch vessel reduces flow into the first side branch vessel by between about 20% and about 30%.

In another example (“Example 7”), further to the method of any one of Examples 5-6, the perfusable portion of the implantable medical device is one or both of the stent element and the graft component.

In another example (“Example 8”), further to the method of Example 7, the method includes arranging the perfusable portion includes arranging a perfusable portion of the graft component adjacent to the first side branch vessel.

In another example (“Example 9”), further to the method of Example 7, the method includes arranging the perfusable portion includes arranging a perfusable portion of the stent component adjacent to the first side branch vessel.

In another example (“Example 10”), further to the method of any one of Examples 1-9, the method includes arranging the implantable medical device to restrict blood flow within the first side branch vessel includes covering the first side branch vessel arranged proximal to the one or more arteries supplying the organ of the patient.

In one example (“Example 11”), an implantable medical device for altering blood flow in a vessel of a patient includes a stent element; and a graft component having attached to at least a portion of the stent element, the graft component having a porosity configured to reduce flow into a first side branch arranged adjacent to the graft component by between about 10% and about 30% to increase flow or pressure at renal arteries of the patient to improve kidney perfusion and diuresis.

In another example (“Example 12”), further to the device of Example 11, the graft component includes a porous film configured to allow blood flow through the film with minimal pressure drop such that flow is reduced between about 10% and about 30% within the vessel.

In another example (“Example 13”), further to the device of Example 11, the graft component includes holes configured to allow blood flow through the film.

In another example (“Example 14”), further to the device of 11, the holes are laser-drilled holes in the graft component.

In one example (“Example 15”), an implantable medical device for altering blood flow in a vessel of a patient includes a stent element configured apply an amount of restriction within the stent element to alter the blood flow within the vessel to increase blood flow into one or more branch vessels extending from the vessel and modify the amount of restriction in response to pulsatile flow; and an anchor portion configured to engage a vessel wall of the vessel and arrange the stent element within the vessel.

In another example (“Example 16”), further to the device of Example 15, the anchor portion includes a membrane component arranged about a portion of the stent element.

In another example (“Example 17”), further to the device of any one of Examples 15-16, the device also includes a restriction portion including a restriction membrane component arranged about a portion of the stent element, the restriction membrane component being configured to restrict a portion of the stent element and taper the stent element and reduce a diameter of the stent element from a proximal end to a distal end.

In another example (“Example 18”), further to the device of Example 17, the restriction membrane component is arranged at the distal end of the stent element, and the anchor portion is at the proximal end of the stent element.

In another example (“Example 19”), further to the device of any one of Examples 15-18, the stent element is configured to lengthen in response to pressure from the pulsatile flow and contract in response to a lack of the pressure, ensuring pressure and increased blood flow to the side branches throughout the entire cardiac cycle.

In another example (“Example 20”), further to the device of any one of Examples 15-19, the vessel is an aorta, and the stent element is configured to increase flow into renal arteries of the patient to improve kidney perfusion and diuresis.

In another example (“Example 21”), further to the device of any one of Examples 15-19, the anchor portion is configured to oppose against the vessel wall in the aorta and create a narrowed flow lumen in a conduit located in the aorta distal of one or both renal arteries of between about 40% and about 80% to alter blood flow into the at least one branch vessel of the aorta.

In another example (“Example 22”), further to the device of any one of Examples 15-19, the anchor portion is configured to oppose against the vessel wall in the vena cava create a narrowed flow lumen in the conduit located in the vena cava distal of one or both renal veins of between about 40% and about 90% to and alter blood flow through one or both of the renal veins.

In another example (“Example 23”), further to the device of Example 22, the stent element is configured to drop blood pressure out of one or both of the renal veins to promote blood flow through the kidneys.

In another example (“Example 24”), further to the device of any one of Examples 15-23, the stent element is configured to increase positive pressure to the one or more branch vessels throughout an entire cardiac cycle.

In another example (“Example 25”), further to the device of any one of Examples 15-24, the stent element and the anchor portion are snareable configured to be retrieved.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows an example implantable medical device in accordance with various aspects of the present disclosure.

FIG. 2 shows an example implantable medical device in accordance with various aspects of the present disclosure.

FIG. 3 shows an example implantable medical device implanted in a patient's vessel in accordance with various aspects of the present disclosure.

FIG. 4 shows a close-up view of a portion of an example implantable medical device in accordance with various aspects of the present disclosure.

FIG. 5 shows an example implantable medical device that alters restriction in accordance with various aspects of the present disclosure.

FIG. 6 shows an example implantable medical device that as altered in response to pulsatile flow in accordance with various aspects of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward treating heart failure in a patient and/or other cardiovascular diseases such as hypertension and hypotension. In certain instances, the condition of the patient may deteriorate by buildup of excess fluid (e.g., hypervolemia) in the body. The buildup of fluid may increase fluid accumulation, principally in the tissues, and increase fluid and pressure in the various circulations and organs. The increased fluid and pressure in and of itself or in combination with an already failing heart may further harm the patient. As discussed in further detail below, various aspects of the present discourse are directed toward lessening buildup of excess fluid by use of an implantable medical device.

Various aspects of the disclosure are directed toward an implantable medical device configured to manipulate renal blood flow hemodynamics in order to induce a physiologically mediated therapeutic response. The implantable medical device discussed herein, in certain instances, is intended to increase natural diuresis and lessen buildup of excess fluid by increasing blood flow to the kidneys. By this action, this device is configured to redirect blood flow to the kidneys to reperfuse the kidneys, improve diuresis (increase fluid removal) and minimize/eliminate the impact of fluid overload on the heart. Kidney health may include the amount of injury that the kidney has sustained, is continuing to sustain, or a decrease in function relative to the baseline kidney function of a patient when healthy. In certain instances, kidney injury may be quantified by measuring Neutrophil gelatinase-associated lipocalin (NGAL).

The implantable medical devices, in certain instances, enable continuous and controlled fluid removal. As explained in further detail below, the patient's vessel near the renal arteries is complex. More specifically, the patient's vessel may include side branches from the aorta in addition to the renal arteries. The implantable medical devices may include a portion of the device that is porous or perfusable to blood flow. In certain instances, the entire device is porous or perfusable to blood flow. In addition, portions of the implantable medical devices may have different porosity or perf usability than other portions of the implantable medical devices. In each of these instances, the implantable medical devices are implanted into a main vessel and are configured to alter blood flow into a side branch from the main vessel.

In certain instances, the implantable medical devices discussed herein may be implanted in other vessels. The implantable medical devices may facilitate increase in peripheral resistance to treat decreases in blood pressure or resistance within the vasculature. As discussed further below, this may include implantation of the implantable medical devices for treatment of an arteriovenous (AV) fistula.

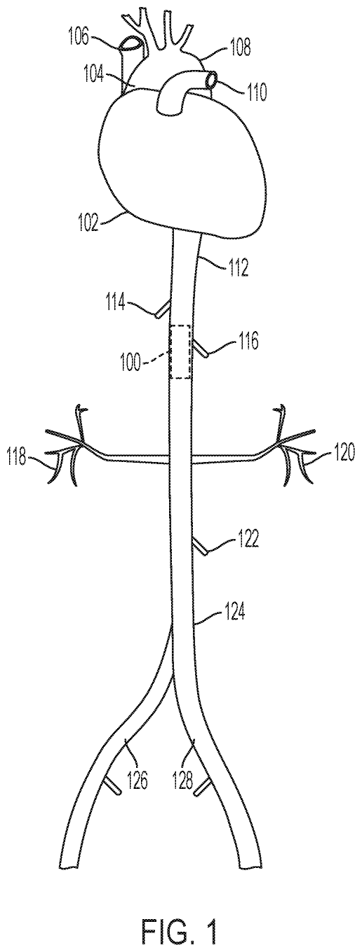

FIG. 1 shows an example implantable medical device 100 in accordance with various aspects of the present disclosure. The implantable medical device 100 is shown arranged within a patient's vasculature. The patient's vasculature shown in FIG. 1 includes the patient's heart 102, aortic root 104, superior vena cava 106, aortic arch 108, pulmonary trunk 110, descending aorta 112, celiac artery 114, superior mesenteric artery 116, renal arteries 118, 120, inferior mesenteric artery 122, abdominal aorta 124, and iliac arteries 126, 128. The implantable medical device 100 may be arranged within the aorta proximal of the renal arteries 118, 120 as shown. In addition, the implantable medical device 100 may be configured to increase blood flow into at least one of the renal arteries 118, 120 while maintaining a substantially unrestricted blood flow within the aorta proximal to the renal arteries 118, 120. As shown, the implantable medical device 100 covers the superior mesenteric artery 116, while allowing less flow into the superior mesenteric artery 116, to increase blood flow into the renal arteries 118, 120. In certain instances, the implantable medical device 100 may be arranged to restrict flow into an artery that are proximal to arteries into which increased blood flow is targeted.

In certain instances, the implantable medical device 100 may be for augmenting perfusion of a branch vessel (e.g., renal arteries 118, 120 or iliac arteries 126, 128) originating from the aorta. The implantable medical device 100 may be adjusted by increasing resistance to blood flow through the implantable medical device 100 to increase pressure within the aorta to increase blood flow into the branch vessel. In addition, the implantable medical device 100 may be configured to remain within the aorta for continuously augmenting perfusion.

The implantable medical device 100 being configured to increase blood flow into at least one of the renal arteries 118, 120 may reduce fluid accumulation by increasing the amount of blood that is filtered by the kidneys. In a patient suffering from heart failure, fluid overload may be caused (at least in part) by insufficient blood flow through the kidneys resulting from compromised cardiac output and venous congestion. Use of the implantable medical device 100 to increase blood flow into at least one of the renal arteries 118, 120 may increase kidney perfusion hemodynamically rather than pharmaceutically. The increased kidney perfusion enhances renal filtration and therefore removes fluid volume. In addition, the implantable medical device 100 may be used to enhance the performance of pharmacological treatments taken in connection therewith. For example, pharmacological treatments (e.g., diuretics and/or hypertensive medications) may be enhanced by additionally enhancing the patient's kidney function.

In certain instances, the implantable medical device 100 being configured to increase blood flow into at least one of the renal arteries 118, 120 while maintaining a substantially unrestricted blood flow within the aorta proximal to the renal arteries 118, 120 may focus blood flow into the one or both of the renal arteries 118, 120. The restriction proximal to the renal arteries 118, 120 may direct blood flow to other areas supplied by the aorta such as the celiac artery 114, the superior mesenteric artery 116, or the brain. Thus, in certain instances, the implantable medical device 100 may be arranged within the aorta of the patient proximal of (or overlapping) arteries proximal to the renal arteries 118, 120. The result may be increased blood flow to at least one of the kidneys, by way of the increased blood flow to one or both of the renal arteries 118, 120, which may increase fluid removal from the circulation which would relieve the fluid and pressure accumulation in the various circulations and organs.

The implantable medical device 100 provides a non-pharmaceutical approach to increasing urine production (diuresis) and/or modifying systemic blood pressure. Patients may experience drug resistance, inaccurate dosing, or undesirable side effects. When drugs fail, aquapheresis or hem odialysis may be used to filter fluid directly from blood, however, these solutions are relatively invasive and disruptive to patient lifestyle and mobility. In addition, aquapheresis or hemodialysis may also produce hemodynamic instability with related cardiovascular complications, kidney damage, infection, and/or require capital equipment.

The implantable medical device 100 may change peripheral resistance when implanted percutaneously or surgically, temporarily or permanently, and may be adjustable to meet patient needs. The implantable medical device 100 may remain in the body after implantation for as long as the patient requires intervention. The implantable medical device 100 may be implanted for hours, days, or even years.

Paired branch vessels that come off the aorta can be angled or not-perpendicular to the aorta. In addition, branch vessels that stem from the aorta that are not paired do not always branch at the same angle (e.g., vessels extending off the aorta are patient anatomy specific). As discussed in further detail below, the implantable medical devices discussed may be arranged to at least partially overlap arteries proximal to the renal arteries 118, 120. The implantable medical devices discussed herein allow lateral blood flow to the arteries in which the implantable medical device overlap to decrease blood flow into those arteries. The result may be increased blood flow to at least one of the kidneys, by way of the increased blood flow to one or both of the renal arteries 118, 120, which may increase fluid removal from the circulation which would relieve the fluid and pressure accumulation in the various circulations and organs. The implantable medical devices allow lateral blood perfusion over the length of the device in order to address implantability difficulties due to branch vessel geometry within a specific patient's anatomy.

The illustrative implantable medical device 100 shown in FIG. 1 is not intended to suggest any limitation as to the scope of use or functionality of embodiments of the disclosure disclosed throughout this document. Neither should the illustrative implantable medical device 100 be interpreted as having any dependency or requirement related to any single component or combination of components illustrated therein. Additionally, any one or more of the components depicted in FIG. 1 can be, in embodiments, integrated with various ones of the other components depicted therein (and/or components not illustrated).

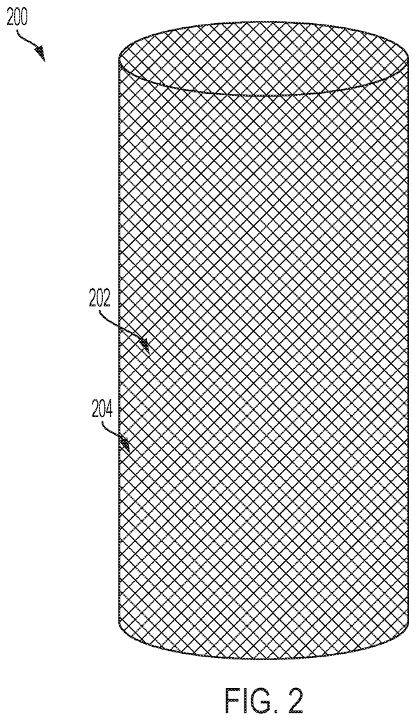

FIG. 2 shows an example implantable medical device 200 in accordance with various aspects of the present disclosure. The implantable medical device 200 is configured for altering blood flow in a vessel of a patient as noted in detail above. The implantable medical device 200 includes a stent element 202 and a graft component 204 attached to at least a portion of the stent element 202.

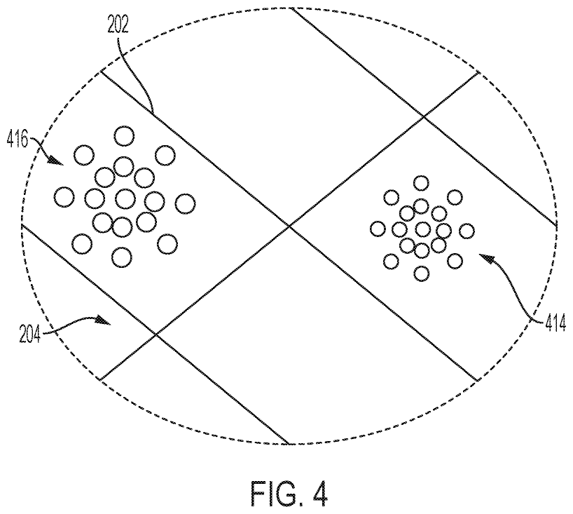

In certain instances, the graft component 204 is at least partially perfusable to allow flow into the side branches while maintaining flow through vessel. The graft component 204 may include pores, as described in further detail below with reference to FIG. 4, that are configured to allow blood flow through the graft component 204. In certain instances, the graft component 204 includes a porous film configured to allow blood flow through the film with minimal pressure drop in within the vessel.

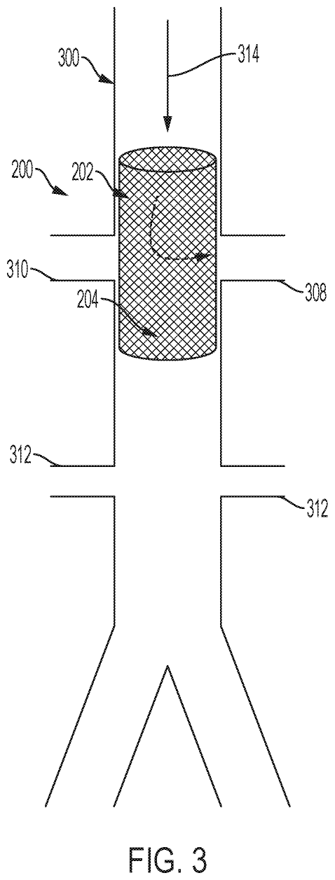

As noted above and described in further detail below with reference to FIG. 3, the implantable medical device 200 may be configured to implant within a patient's aorta and restrict or increase flow into at least one of the celiac, the hepatic, and the mesenteric arteries. In certain instances, the implantable medical device 200 may increase or decrease flow into a branch vessel (or vessel pairs) that may include gastric, splenic, adrenal (paired), phrenic (paired), gonadal (paired), lumbar (paired) and sacral (unpaired) arteries.

When implanted in the aorta, the device 200 is configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the device 200 is configured to create a narrowed flow lumen in the conduit of the aorta of the patient at least partially distal of the renal arteries between about 40% and about 80% and alter blood flow into at least one branch vessel of the aorta (e.g., one or both of the renal arteries). In certain instances, the induced restriction is between about 50% and about 70% of a nominal flow.

When implanted in the vena cava, the device 200 may augment perfusion from a tributary vessel (e.g., renal veins) terminating in the vena cava by altering pressure within the vena cava to alter blood flow from the tributary vessel of the vena cava. In certain instances, the device 200 may be configured to create a narrowed flow lumen in the conduit located in the vena cava distal of the at least one tributary vessel of between about 40% and about 90%. Use of the flow restriction devices, discussed in further detail below, by dropping pressure in the renal veins may increase kidney perfusion hemodynamically rather than pharmaceutically.

In certain instances, the device 200 is arranged in a vessel other than the aorta or venal cava. In these instances, the device 200 may be configured to alter the blood flow through the lumen to restrict blood flow in the vessel and induce a physiologically mediated therapeutic response in the patient. In certain instances, the device 200 is configured to induce the physiologically mediated therapeutic response to include an increase in peripheral resistance within the vessel. The device 200 may be configured to treat a fistula within the vessel and increase in peripheral resistance within the vessel as described in further detail below.

FIG. 3 shows an example implantable medical device 200 implanted in a patient's vessel 300 in accordance with various aspects of the present disclosure. In certain instances, the implantable medical device 200 may be used in a method of altering blood flow 314 in a vessel of a patient. As shown in FIG. 3, the implantable medical device 200 is implanted within an aorta. The implantable medical device 200, which includes a stent element 202 and a graft component 204 attached to at least a portion of the stent element 202, may be delivered to a target location within the vessel (e.g., the aorta). The implantable medical device 200 is arranged to at least partially direct flow into one or more side branches 308, 310, 312 off the vessel (aorta) 300.

In altering blood flow in the vessel 300, the implantable medical device 200 is delivered within the vessel and arranged to restrict blood flow within one or more first branches 308, 310, vessel to reduce flow into one or both of the first branches 308, 310, to increase flow into one or more arteries (such as vessels 312) supplying an organ of the patient. In certain instances, the organ is one of the adrenal glands, testes or ovaries, pancreas, intestine, appendix, liver, stomach, gallbladder, duodenum, spleen, vertebrae, bladder, or muscles of the patient. The implantable medical device 200, as explained in detail below, is arranged to restrict blood flow into the first side branch vessel or vessels 308, 310 that is proximal to the one or more arteries 312 supplying the organ of the patient.

In instances where the organ is the kidneys, the branch into which greater flow is intended is one or more of the renal arteries. By arranging the implantable medical device 200 in this manner, the graft component 204 and/or the stent component 202 restrict blood flow within the one or more branches 308, 310 to increase flow into one or more arteries (such as vessels 312). In certain instances, the vessels 312 may be a renal artery with increased flow into one or both renal arteries of the patient improve kidney perfusion. In certain instances, the graft component 204 and/or the stent component 202 are configured to restrict blood flow to the first side branch vessel or vessels 308, 310 by covering the first side branch vessel or vessels 308, 310 arranged proximal to a renal artery ostia.

To reduce blood flow into the side branch vessel or vessels 308, 310, a perfusable portion of the implantable medical device 200 is arranged adjacent to the first side branch vessel or vessels 308, 310. In certain instances, the entire implantable device 200 is perfusable and allows blood flow laterally. The blood flow does not laterally flow from the implantable device 200 in portions due to the implantable device 200 contacting the vessel 300 wall. The perfusable portion, the portion arranged adjacent to the first side branch vessel or vessels 308, 310, reduces flow into the first side vessel or vessels 308, 310 by approximately about 10%—about 20%, about 20%—about 30%, about 30%—about 40% or any number therebetween. In certain instances, the graft component 204 is perfusable and therefore controls the amount of blood flow into the branch vessel or vessels 308, 310. The porosity of the graft component 204 may be tailored to achieve the desired amount of flow reduction into the vessel or vessels 308, 310. In other instances, the stent component 202 is perfusable and controls the amount of flow into the vessel or vessels 308, 310. The stent component 202 may be weaved or arranged to achieve the desired amount of flow reduction into the vessel or vessels 308, 310. Further, the combination of the graft component 204 and the stent element 202, in other instances, may control the amount of flow into the vessel or vessels 308, 310. In certain instances, the porosity of the graft component 204 and the weave or arrangement of the stent component 202 is tailored to achieve the desired amount of flow reduction into the vessel or vessels 308, 310. The stent component 202 may be a wound wire structure or laser cut from a tube.

In certain instances, the graft component 204 of the implantable medical device 200 includes one or more porous or perfusable portions. The implantable medical device 200 may be arranged such that the porous or perfusable portions of the graft component 204 of the implantable medical device 200 are arranged adjacent to the one or more side branches 308, 310, 312 to direct flow into one or more side branches 308, 310, 312 that are distal to the branch vessels 308, 310, 312 by which the implantable medical device 200 is arranged. The implantable medical device 200 may alter pressure within the aorta to increase or decrease blood flow 314 into the side branches 308, 310, 312 (such as the renal arteries). An increase of pressure at or distal to the renal arteries 118, 120 or targeted branch(es) such as the iliac arteries 126, 128 by the implantable medical device 200 (shown above in FIG. 1) may increase blood flow 314 into areas distal thereto (e.g., into the renal arteries 118, 120 and/or the iliac arteries 126, 128 depending on the placement of the implantable medical device 200).

In certain instances, the implantable medical device 200 may produce a long-term or chronic physiological change in the patient. The implantable medical device 200 alters flow into the kidneys and may produce a neuro-hormonal response that effects a change in the patient to move toward normal kidney functioning. The kidneys are a feedback regulator of systemic pressure through the patient's body. The implantable medical device 200 alters flow into the kidneys and provides a non-pharmaceutical means of influencing the kidneys' natural feedback mechanisms to regulate systemic pressure.

By adjusting the degree of hemodynamic alteration of renal perfusion, patient-specific adjustments to regulate blood pressure may be made. Adjusting the aortic fluid flow rate imparted by the implantable medical device 200, may influence renal artery pressure and/or flow rate, which, in turn, can manifest as transient or long-lasting alterations in systemic blood pressure. The changes induced by the implantable medical device 200, in renal-mediated blood pressure levels, may have therapeutic benefits in and of themselves. Likewise, changes induced by the implantable medical device 200 in renal-mediated blood pressure levels may be used in combination with various blood pressure medications to optimize blood pressure management on an individualized basis.

The implantable medical device 200 may occlude the branches proximal to the side branches 308, 310 (e.g., renal arteries) by between about 5% and about 30% to increase blood flow 314 into the kidneys. The porosity of the implantable medical device 200 may be tailored to achieve between about 5% and about 30% to increase of blood flow 314 into the kidneys. As is explained in further detail below with reference to FIG. 4, the size, location, number, and perfusability of the pores may be altered to achieve the desired blood flow.

FIG. 4 shows a close-up view of a portion of an example implantable medical device 200 in accordance with various aspects of the present disclosure. As shown in FIG. 4, the graft component 204, coupled to a stent component 202, may include pores 414 that are configured to allow blood flow through the film. The pores 414 may be perforations or laser-drilled holes in the graft component 204. The openings or pores 414 in the graft component 204 can further provide perfusion to a side branch vessel. For example, the graft component 204 can have a perfusion region with pores 414 and an excluding region substantially without the pores 414.

As shown in FIG. 4, the graft component 204 may include additional pores 416 that have a different porosity than the pores 414. The additional pores 416 and the pores 414 may be of different size, shape, and/or location. As a result and in certain instances, the graft component 404 includes a first portion having a porosity configured to direct flow into one or more side branches off the vessel by about 10%—about 20%, about 20%—about 30%, about 30%—about 40% or any number therebetween to improve kidney perfusion and diuresis and a second component that is non-porous and configured to inhibit blood flow radially through the second component. In addition, the graft component 204 may include multiple layers that have different porosity.

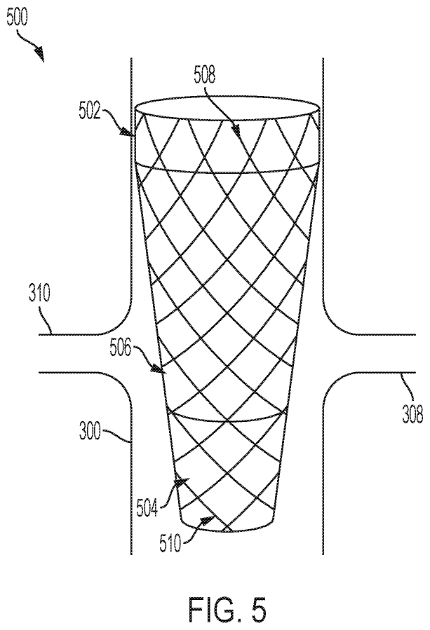

FIG. 5 shows an example implantable medical device 500 that alters restriction in accordance with various aspects of the present disclosure. The implantable medical device 500 includes a stent element 506 and an anchor portion 502. The implantable medical device 500 is shown arranged within a patient's vessel 300.

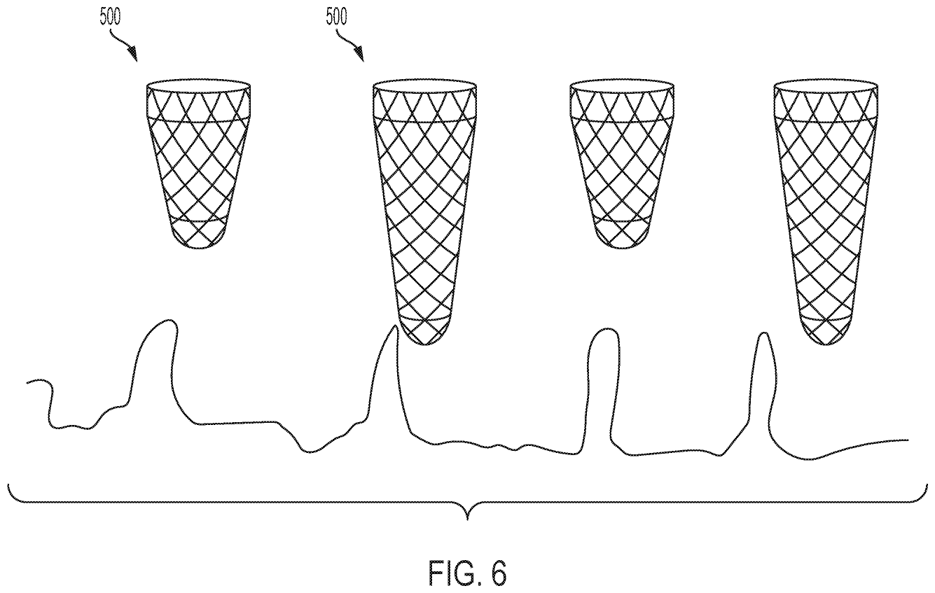

The stent element 506 is configured to apply an amount of restriction to alter the blood flow within the vessel to increase blood flow into one or more branch vessels 308, 310 extending from the vessel 300 and modify the amount of restriction in response to pulsatile flow as shown and discussed in further detail with reference to FIG. 6. The stent element 506 is configured to lengthen in response to pressure from the pulsatile flow and contract in response to a lack of the pressure. In addition, the vessel 300 is an aorta, and the stent element 506 is configured to increase flow into renal arteries of the patient to improve kidney perfusion and diuresis. In certain instances, the stent element 506 is configured to lengthen in response to pressure from the pulsatile flow and contract in response to a lack of the pressure, ensuring pressure and increased blood flow to the side branches throughout the entire cardiac cycle.

The anchor portion 502 of the implantable medical device 500 is configured to engage a vessel wall of the vessel 300 and arrange the stent element 500 within the vessel 300. The anchor portion 502 can include a membrane or graft component 508 that lessens the opportunity for thrombosis. The membrane or graft component 508 of the anchor portion 520 may contact the vessel wall.

In certain instances, the implantable medical device 500 includes a restriction portion 504 including a restriction membrane component 510 arranged about a portion of the stent element 506. The restriction membrane component 510 is configured to restrict a portion of the stent element 506 and taper the stent element and reduce a diameter of the stent element 506 from a proximal end to a distal end. As shown, the restriction membrane component 510 is arranged at the distal end of the stent element 506, and the anchor portion 502 is at the proximal end of the stent element 506.

In certain instances and as shown, the stent element 506 may partially occlude side branches (e.g., proximal to renal arteries) 308, 310 by approximately between about 5% and about 30% to increase blood flow into the kidneys. The implantable medical device 500 may be implanted to have lateral perfusion and restrict blood flow into one or more arteries 308, 310 proximal to the renal arteries (or other arteries supplying an organ) to increase blood flow into the renal arteries (or other arteries supplying an organ) that are distal to the location of the implantable medical device 500. In these instances, the stent element 506 is perfusable as discussed in detail above.

In certain instances, the anchor portion 502 is arranged upstream from the renal arteries in in case the landing zone, portions of the stent element 506 between the anchor portion 502 and the restriction membrane component 510, is obstructed. In other instances, the anchor portion 502 may be arranged below the renals with the same clinical effect. The implantable medical device 500 is configured for many placements that are normally plagued with disease or acute artery angulation. In addition, the restriction membrane component 510 facilitates the expansion and contraction of the stent element 506.

In certain instances, when increased flow encounters the implantable medical device 500, there is more flow to the upstream side vessels due to the impediment of flow by the implantable medical device 500. As the implantable medical device 500 elongates, the flow to the renals (or other branch vessels) increases due to the restriction membrane component 510 restricting flow. As the implantable medical device 500 contracts and snaps back upstream, the implantable medical device 500 also forces blood into the renals (or other side branches) due to the implantable medical device 500 pulling a small amount of blood back upstream with the implantable medical device 500. As a result, the implantable medical device 500 increases positive pressure to the renals (or side branches) throughout the entire cardiac cycle. In certain instances, the implantable medical device 500 is snareable or retrievable by a clinician during the procedure or at a later date.

In other instances, the stent element 506 is not perfusable and the implantable medical device 500 is not arranged as shown. In these instances, the stent element 506 may increase or decrease a fluid flow rate, within the vessel (aorta) 300 distal to the side branches (e.g., renal arteries) 308, 310, by between about 5% and about 30% as compared to normal flow. In certain instances, the stent element 506 is configured to induce stenosis of the aorta of the patient at least partially distal of the side branches (e.g., renal arteries) 308, 310 between about 40% and about 80% and alter blood flow into one or more of the side branches (e.g., renal arteries) 308, 310 while maintaining a substantially unrestricted blood flow within the vessel (aorta) 300 proximal to one or more of the side branches (e.g., renal arteries) 308, 310. In certain instances, the induced stenosis is between about 50% and about 70%. Clinically, measurement of ankle pressure, Doppler ultrasound velocity, ankle-brachial index, or other hem odynam is parameters in the lower limbs can be employed to optimize the magnitude of the induced stenosis while ensuring adequate limb perfusion.

When implanted in the aorta, the device 500 is configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the device 500 is configured to create a narrowed flow lumen in the conduit of the aorta of the patient at least partially distal of the renal arteries between about 40% and about 80% and alter blood flow into at least one branch vessel of the aorta (e.g., one or both of the renal arteries). In certain instances, the induced restriction is between about 50% and about 70% of a nominal flow.

When implanted in the vena cava, the device 500 may augment perfusion from a tributary vessel (e.g., renal veins) terminating in the vena cava by altering pressure within the vena cava to alter blood flow from the tributary vessel of the vena cava. In certain instances, the device 500 may be configured to create a narrowed flow lumen in the conduit located in the vena cava distal of the at least one tributary vessel of between about 40% and about 90%. Use of the flow restriction devices, discussed in further detail below, by dropping pressure in the renal veins may increase kidney perfusion hemodynamically rather than pharmaceutically.

FIG. 6 shows an example implantable medical device 500 as altered in response to pulsatile flow in accordance with various aspects of the present disclosure. The pulsatile flow, represented by the R wave in an EKG, is shown adjacent to the implantable medical device 500 contracting and lengthening. The stent element 506 of the implantable medical device 500 is configured to lengthen in response to pressure from the pulsatile flow and contract in response to a lack of the pressure as shown in FIG. 6. The stent element 506 applies further restriction in the lengthen configuration to restrict blood flow into side branch vessels proximal to arteries into which increased blood flow is desired.

In certain instances, patients with heart failure (such as late-stage heart failure) may have an elevated sympathetic nervous system state in part due to decreased cardiac output (blood pressure and flow in one of both of the kidneys). One compensatory output of this state is to generate a signal to attempt to preserve cardiac output, which puts further strain (myocardial oxygen demand) on the heart. Implantable medical devices discussed herein, and the methods that include the implantable medical devices, are directed toward increasing the pressure (mean or peak systolic) in the kidney to reduce stimulation of the neuro-hormonal response (e.g., decrease in the sympathetic activation nervous system). The result of the reduced activation of the sympathetic nervous system, by way of the implantable medical device or methods that include the implantable medical device, may decrease resting heart rate and blood pressure.

Further and in certain instances, patients with heart failure (such as late-stage heart failure) may have activation of the Renin-Angiotensin-Aldosterone system (RAAS), in part due to decreased cardiac output resulting in impaired blood flow to the kidney. A consequence of the elevated RAAS is to generate a signal that stimulates adverse myocardial structural changes. Implantable medical devices discussed herein, and the methods that include the implantable medical devices, are directed toward increasing the pressure (mean or peak systolic) in the kidney to reduce stimulation of the RAAS. The result of the reduced activation of the RAAS, by way of the implantable medical device or methods that include the implantable medical device, may be a reduced sympathetic nervous system activation and attenuation of adverse cardiac remodeling.

Previous studies of implantable medical devices implanted in the aorta have been used to evaluate response of canines with induced heart failure (coronary microembolization resulting in ejection fraction of about 30%). Hemodynamic status observed in the test group relative to the control group indicated improved cardiac function and decreased sympathetic nervous system tone. For example, heart rate and mean arterial pressure decreased, while contractility increased relative to controls. These comparative outcomes were supported by positive shifts in biomarkers such as pro-BNP and NGAL, relative to controls. As an example, animals with implant produced about 35% more urine with about 21% higher creatinine content, resulting in about 52% less increase in serum creatinine as a result of the diuretic challenge. These results show that an implantable medical device, such an implantable medical device directing blood into the kidneys or restricting blood flow within the aorta distal to the renal arteries, placed in the aorta may help decrease the symptoms of fluid overload and cardiac stress associated with heart failure. The devices are shown to increase blood pressure proximal to the stenosis and, in doing so, increase kidney perfusion pressure, thus increasing kidney perfusion. A secondary effect of this device is the reduction in the activation of the RAAS system. Effectiveness of the device was based on assessment of central hemodynamics, left ventricular (LV) function and renal function.

In addition, previous studies have found that induced stenosis has little effect on flow or pressure until it reaches about 40%, after which the impact is dependent on artery diameter and blood flow rate. Based on the above animal study, however, it has been discovered that there is a threshold above which the impact dramatically increases. The regime for stenosis, based on these results, is between about 40% and about 80%, and more particularly between about 50% and about 70%. Clinically, measurement of ankle pressure, Doppler ultrasound velocity, or other hemodynamic parameters in the lower limbs can be employed to optimize the magnitude of the induced stenosis while ensuring adequate limb perfusion.

In addition and in certain instances, the devices discussed above describe diametric restriction of the aorta or vena cava, however, a length of the restriction portion also may affect the amount of restriction in the aorta or vena cava. Thus, the length of the restriction portion and the diameter or circumference of the restriction portion may be varied to achieve a desired stenosis or restriction percentage.

Further, the devices discussed herein may implanted within vessels for treatment of an arteriovenous (AV) fistula. AV fistula formation may lead to a decrease in peripheral resistance within the vasculature. Implantation of the devices discussed herein at or adjacent to the AV fistula may increase flow resistance to a nominal level and counteract the decrease in peripheral resistance resulting from the AV fistula. In certain instances, the devices are implanted distal to the AV fistula, proximal to the AV fistula, or across the AV fistula.

Examples of synthetic polymers (which may be used as a graft component) include, but are not limited to, nylon, polyacrylamide, polycarbonate, polyform aldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyam ides, their mixtures, blends and copolymers are suitable as a graft material. In one embodiment, said graft is made from a class of polyesters such as polyethylene terephthalate including DACRON® and MYLAR ® and polyaram ids such as KEVLAR®, polyfluorocarbons such as polytetrafluoroethylene (PTFE) with and without copolymerized hexafluoropropylene (TEFLON®. or GORE-TEX®.), and porous or nonporous polyurethanes. In certain instances, the graft comprises expanded fluorocarbon polymers (especially PTFE) materials described in British. Pat. No. 1,355,373; 1,506,432; or 1,506,432 or in U.S. Pat. Nos. 3,953,566; 4,187,390; or 5,276,276, the entirety of which are incorporated by reference. Included in the class of preferred fluoropolymers are polytetrafluoroethylene (PTFE), fluorinated ethylene propylene (FEP), copolymers of tetrafluoroethylene (TFE) and perfluoro(propyl vinyl ether) (PFA), homopolymers of polychlorotrifluoroethylene (PCTFE), and its copolymers with TFE, ethylene-chlorotrifluoroethylene (ECTFE), copolymers of ethylene-tetrafluoroethylene (ETFE), polyvinylidene fluoride (PVDF), and polyvinyfluoride (PVF). Especially preferred, because of its widespread use in vascular prostheses, is ePTFE. In certain instances, the graft comprises a combination of said materials listed above. In certain instances, the graft is substantially impermeable to bodily fluids. Said substantially impermeable graft can be made from materials that are substantially impermeable to bodily fluids or can be constructed from permeable materials treated or manufactured to be substantially impermeable to bodily fluids (e.g. by layering different types of materials described above or known in the art).

Additional examples of graft materials include, but are not limited to, vinylidinefluoride/hexafluoropropylene hexafluoropropylene (HFP), tetrafluoroethylene (TFE), vinylidenefluoride, 1-hydropentafluoropropylene, perfluoro(methyl vinyl ether), chlorotrifluoroethylene (CTFE), pentafluoropropene, trifluoroethylene, hexafluoroacetone, hexafluoroisobutylene, fluorinated poly(ethylene-co-propylene (FPEP), poly(hexafluoropropene) (PHFP), poly(chlorotrifluoroethylene) (PCTFE), poly(vinylidene fluoride (PVDF), poly(vinylidene fluoride-co-tetrafluoroethylene) (PVDF-TFE), poly(vinylidene fluoride-co-hexafluoropropene) (PVDF-HFP), poly(tetrafluoroethylene-co-hexafluoropropene) (PTFE-HFP), poly(tetrafluoroethylene-co-vinyl alcohol) (PTFE-VAL), poly(tetrafluoroethylene-co-vinyl acetate) (PTFE-VAC), poly(tetrafluoroethylene-co-propene) (PTFEP) poly(hexafluoropropene-co-vinyl alcohol) (PHFP-VAL), poly(ethylene-co-tetrafluoroethylene) (PETFE), poly(ethylene-co-hexafluoropropene) (PEHFP), poly(vinylidene fluoride-co-chlorotrifluoroe-thylene) (PVDF-CTFE), and combinations thereof, and additional polymers and copolymers described in U.S. Publication 2004/0063805, incorporated by reference herein in its entirety for all purposes. Additional polyfluorocopolymers include tetrafluoroethylene (TFE)/perfluoroalkylvinylether (PAVE). PAVE can be perfluoromethylvinylether (PMVE), perfluoroethylvinylether (PEVE), or perfluoropropylvinylether (PPVE), as essentially described in U.S. Publication 2006/0198866 and U.S. Pat. No. 7,049,380, both of which are incorporated by reference herein for all purposes in their entireties. Other polymers and copolymers include, polylactide, polycaprolacton-glycolide, polyorthoesters, polyanhydrides; poly-aminoacids; polysaccharides; polyphosphazenes; poly(ether-ester) copolymers, e.g., PEO-PLLA, or blends thereof, polydimethyl-siolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters and any polymer and copolymers described in U.S. Publication 2004/0063805, incorporated by reference herein in its entirety.

The graft components, as discussed herein, may be attached to the self-expanding stent elements by using a coupling member that is generally a flat ribbon or tape having at least one generally flat surface. In certain instances, the tape member is made from expanded PTFE (ePTFE) coated with an adhesive. The adhesive may be a thermoplastic adhesive. In certain instances, the thermoplastic adhesive may be fluorinated ethylene propylene (FEP). More specifically, an FEP-coated side of the ePTFE may face toward and contacts an exterior surface of the self-expanding stent and graft component, thus attaching the self-expanding stent to the graft component. Materials and method of attaching a stent to the graft is discussed in U.S. Pat. No. 6,042,602 to Martin, incorporated by reference herein for all purposes.

The stent elements discussed herein can be fabricated from a variety of biocompatible materials. These materials may include 316L stainless steel, cobalt-chromium-nickel-molybdenum-iron alloy (“cobalt-chromium”), other cobalt alloys such as L605, tantalum, nickel-titanium alloys (e.g., Nitinol), or other biocompatible metals. In certain instances, as discussed in detail above, the stent (and graft) may be self-expanding. The prosthesis may be balloon expandable

A variety of materials variously metallic, super elastic alloys, such as Nitinol, are suitable for use in these stents. Primary requirements of the materials are that they be suitably springy even when fashioned into very thin sheets or small diameter wires. Various stainless steels which have been physically, chemically, and otherwise treated to produce high springiness are suitable as are other metal alloys such as cobalt chrome alloys (e.g., ELGILOY®), platinum/tungsten alloys, and especially the nickel-titanium alloys (e.g., Nitinol).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. An implantable medical device for altering blood flow in a vessel of a patient, the device comprising:
a stent element; and
a graft component having attached to at least a portion of the stent element, the graft component having a porosity configured to reduce flow into a first side branch arranged adjacent to the graft component by between about 10% and about 30% to increase flow or pressure at renal arteries of the patient to improve kidney perfusion and diuresis.

2. The device of claim 1, wherein the graft component includes a porous film configured to allow blood flow through the film with minimal pressure drop such that flow is reduced between about 10% and about 30% within the vessel.

3. The device of claim 1, wherein the graft component includes holes configured to allow blood flow through the film.

4. The device of claim 1, wherein the holes are laser-drilled holes in the graft component.

5. An implantable medical device for altering blood flow in a vessel of a patient, the device comprising:
a stent element configured apply an amount of restriction within the stent element to alter the blood flow within the vessel to increase blood flow into one or more branch vessels extending from the vessel and modify the amount of restriction in response to pulsatile flow; and
an anchor portion configured to engage a vessel wall of the vessel and arrange the stent element within the vessel.

6. The device of claim 5, wherein the anchor portion includes a membrane component arranged about a portion of the stent element.

7. The device of any one of claim 5, further comprising a restriction portion including a restriction membrane component arranged about a portion of the stent element, the restriction membrane component being configured to restrict a portion of the stent element and taper the stent element and reduce a diameter of the stent element from a proximal end to a distal end.

8. The device of claim 7, wherein the restriction membrane component is arranged at the distal end of the stent element, and the anchor portion is at the proximal end of the stent element.

9. The device of claim 5, wherein the stent element is configured to lengthen in response to pressure from the pulsatile flow and contract in response to a lack of the pressure, ensuring pressure and increased blood flow to the side branches throughout the entire cardiac cycle.

10. The device of claim 5, wherein the vessel is an aorta, and the stent element is configured to increase flow into renal arteries of the patient to improve kidney perfusion and diuresis.

11. The device of claim 5, wherein the anchor portion is configured to oppose against the vessel wall in the aorta and create a narrowed flow lumen in a conduit located in the aorta distal of one or both renal arteries of between about 40% and about 80% to alter blood flow into the at least one branch vessel of the aorta.

12. The device of claim 5, wherein the anchor portion is configured to oppose against the vessel wall in the vena cava create a narrowed flow lumen in the conduit located in the vena cava distal of one or both renal veins of between about 40% and about 90% to and alter blood flow through one or both of the renal veins.

13. The device of claim 12, wherein the stent element is configured to drop blood pressure out of one or both of the renal veins to promote blood flow through the kidneys.

14. The device of claim 5, wherein the stent element is configured to increase positive pressure to the one or more branch vessels throughout an entire cardiac cycle.

15. The device of claim 5, wherein the stent element and the anchor portion are snareable configured to be retrieved.

16. A method of altering blood flow in a vessel of a patient, the method com prising:
delivering an implantable medical device including a stent element and a graft component attached to at least a portion of the stent element within the vessel; and
arranging the implantable medical device to restrict blood flow within a first side branch vessel to reduce flow into the first side branch and increase flow into one or more arteries distal to the first side branch vessel and supplying an organ of the patient.

17. The method of claim 16, wherein the organ is one of kidneys, brain, pancreas, or liver.

18. The method of claim 16, wherein arranging the implantable medical device to restrict blood flow within the first side branch vessel includes reducing flow into the first side branch to increase flow into one or both renal arteries of the patient improve kidney perfusion.

19. The method of claim 17, wherein arranging the implantable medical device to restrict blood flow within the first side branch vessel includes covering the first side branch vessel arranged proximal to a renal artery ostia

20. The method of claim 16, wherein arranging the implantable device includes arranging a perfusable portion of the implantable medical device adjacent to the first side branch vessel.
